(19) United States
(12) Patent Application Publication
(10) Pub. No.: US 2005/0240154 A1
(43) Pub. Date: Oct. 27, 2005

(54) Infusion set with patch

(75) Inventors: Lasse W. Mogensen, Soborg (DK); Grete Kornerup, Vipperod (DK)

Correspondence Address: BRINKS HOFER GILSON & LIONE, P.O. BOX 10395, CHICAGO, IL, 60610, US

(73) Assignee: Unomedical A/S:

(21) Appl. No.: 11/110,305

(22) Filed: Apr. 20, 2005

Related U.S. Application Data
(60) Provisional application No. 60/563,919, filed on Apr. 21, 2004.

(51) Int. Cl. A61M 5/00

(52) U.S. Cl. 604/173

(57) ABSTRACT

An infusion set including an infusion part and a connector being in fluid communication with a medical device is provided. The infusion part includes multiple micro needles being in fluid communication with a chamber and the chamber is supplied with fluid from the medical device. The fluid is conducted through a conduit which opens into the chamber and the chamber is in fluid contact with the inlets of all micro needles.

This application claims the benefit of U.S. Provisional Application Ser. No. 60/563,919, filed Apr. 21, 2004, which is hereby incorporated by reference in its entirety.

### THE TECHNICAL FIELD

The invention relates to an infusion set for an intermittent or continuous administration of a therapeutical substance, such as insulin. A traditional infusion set comprises an infusion part having multiple micro needles and being in fluid communication with a medical device preferably a medical delivery device such as an insulin pump.

### BACKGROUND

U.S. Pat. No. 6,629,949 B1 discloses as system for delivery of a medicament to be used with an infusion pump, the delivery device having a plurality of projections and a drug reservoir. The construction according to U.S. Pat. No. 6,629,949 B1 does not allow the patient to disconnect from the infusion pump. Further administration via such a system has the disadvantage that micro needles which are placed close to the drug inlet administer a larger part of the drug (actually most of the drug) than micro needles placed far from the inlet. This can give an inexpedient delivery profile.

US 2003/0181863 relates to an adapter for transport of fluids with a micro needle device. The adapter can receive for example a syringe or the infusion part of an infusion set, and then fluid is transported through the adapter and applied into a patient using the micro needle device. FIG. 7 of this publication illustrates how the adapter can be used in conjunction with an infusion set (50) where the infusion set body (52) is connected to an adapter (60). The adapter include an aperture (68) formed at a shallow angle relative to a body (62) of the adapter (60). The adapter include a micro needle array (66) formed at the end of a housing (62) having an angle which is non-parallel to the long axis of the housing. FIG. 7 shows how the fluid is transferred directly from a cannula (56) which is unreleasably connected to the infusion part of an infusion set to a cavity limited by the septum (64) and the upper side of the microneedle array (66) which is in fluid contact with the microneedles.

Thus the object of the present invention is to provide an infusion set for an intermittent or continuous administration of a therapeutical substance, which allows a person to move around.

### DESCRIPTION OF THE PRESENTLY PREFERRED EMBODIMENTS

According to the invention there is provided an infusion set comprising an infusion part and a connector being in fluid communication with a medical device. The infusion part comprises multiple micro needles which are in fluid communication with a chamber, which chamber is supplied with fluid from the medical device characterized in that fluid is conducted through a conduit which opens into the chamber, and the chamber is in fluid contact with the inlets of all microneedles.

The infusion set described above is capable of being connected/disconnected from the medical device by means of the connector thus allowing the person to move around and to reconnect later when new administration is needed. Also this system supplies the drug through the micro needles thus giving a fast distribution of the drug in the patient.

The infusion part of this infusion set is constructed in such manner that the pressure drop from the drug supply to all the inlets essentially is the same.

When the pressure of the liquid is the same at the inlets, then the flow through the needle is identical and therefore an equal amount of drug flows through each needle.

In a preferred embodiment the chamber which functions as a distribution chamber has rigid walls where “rigid walls” means that the walls in the chamber will not move, give in or change shape as a result of the fluid pressure produced by the injected fluid.

This new system has the advantage that there is no need for an insertion device since the infusion part only has to be placed on the skin of the carrier and then it is ready for use. This is especially helpful for people who feel discomfort during self-administration since they no longer has to insert large cannulas.

The area carrying the micro needles can be an integral part of the infusion part or preferably in form of a patch attached to the rest of the infusion part i.e. the micro needles is placed at a separate patch. The type of micro needles used in the present invention is by no means critical; in fact any known micro needle arrays can be used.

In a preferred embodiment the micro needles have one of the following elaborations: a cone having a side outlet i.e. the outlet is placed on the side of the cone, a hollow needle with outlets on the side of the cylindrical part, a cone with outlet at the tip, a needle having an outlet at its foot of attachment or next to its attachment point, a slashed needle, a needle having prickles thus securing the patch better to the skin and with an outlet either at the bottom, at the side or at the point.

The needles can be made of any non-fluid and biocompatible material.

In a preferred embodiment a biodegradable needle is used and this is especially useful when needles with prickles are used.

Preferred material for the needles is one or more of the following material: pharmaceutical grade stainless steel, gold, titanium, nickel, iron, silver, platinum, tin, copper, chromium, alloys of previous metals, silicon, silicon dioxide, ceramics, acrylic materials and polymers such as polycarbonate, polymethacrylic acid, ethylenevinyl actetate, polytetrafluorethane, polyesters and surface treated materials. Especially preferred materials are silicon and acrylic materials.

Preferred biodegradable materials are polymers of hydroxylic acids such as lactic acid and glycolic acid, polylactide, polyglycolide, polylactide-co-glycolide, polyanhydrides, pole(ortho)esters, polyurethanes, poly(butyric acid), poly(valeric acid) and poly (lactide-co-caprolactone).

In a preferred embodiment the micro needles has a length between 50 and 500 μm. Preferably the needles has a diameter from 10 to 300 μm and/or preferably a hole less than 300 μm.

In a preferred embodiment the area carrying the micro needles raises itself from the lower surface i.e. it is the area of the lower surface having the largest distance to the main plane of the infusion part. This can be done either by elaborating the lower surface of the infusion part as a hill and placing the micro needles at the top of the hill or it can be done by having a step on the lower surface and placing the micro needles on this step. By having the needle placed on an elevated point it is assured that the needle will touch and penetrate the skin of the carrier and that there is maximum pressure on the area carrying the needles.

In a preferred embodiment the lower surface of the infusion part is convex.

In a preferred embodiment the infusion part is secured on the skin of the carrier either by use of an adhesive member such as a plaster, by having an adhesive layer on the bottom of the infusion part or by use of a bandage. The possibility of using a bandage to secure the infusion part to the skin is an advantageous feature of this new infusion set. There are people who are allergic to adhesives and since ordinary infusion sets are provided with adhesive support, these people are prevented from using the ordinary infusion sets.

In a preferred embodiment the micro needles penetrates the plaster.

In another preferred embodiment the plaster is placed on top of the infusion part and thus covers most of the infusion part when attached to a person.

In a preferred embodiment there is pressure on the area carrying the micro needles.

In the embodiment where a separate patch carrying the micro needles is attached to the infusion part, the patch can be attached by means of taps, rivets, screws, by welding, by adhesives, by heat deformation, by pressure i.e. having a patch slightly larger than the hole receiving it optionally said hole having elastic walls or by bending a tap over the edge of the patch.

In a preferred embodiment the infusion part further comprises a packing. The packing secures that in case the needles leak and the drug remain in the desired area and does not leak into the plaster. Preferably the packing is in form of a flange or a ring preferably surrounding the needles.

In a preferred embodiment the infusion set further comprises a leak detector, preferably in form of a plaster changing its colour in case of leakage.

In a preferred embodiment the infusion set further comprises an electrode for electrical stimulation of the drug receiving area, thus providing a better transport of the drug across the barrier.

In one embodiment of the invention a part of the chamber is split up in multiple canals each forming the fluid communication between the chamber and the micro needles, and the canals having their length and diameter adjusted to each other in such manner that the fluid pressures at the inlet to the micro needles are essentially the same or creates a desired pressure profile in the chamber.

In another construction the chamber has its largest thickness measured perpendicular to the main plane at the inlet to the chamber and then have a decreasing thickness from this point and to the edges of the chamber. The decrease in thickness is adjusted in such manner that the pressure at the inlet of the needles essentially is the same for all needles.

In a preferred embodiment the chamber has supporting members which prohibit a blockage of the chamber or a sealing off of a part of the chamber.

In another embodiment the chamber is filled with a transport material which facilitates transport of fluid through the chamber. Examples of such materials are a hard material having grooves providing fluid communication through the chamber, a hard material having cross-millings, felt, a porous material, cotton, silicon or silicon oxides materials, blown materials and micro cellular urethane materials. Suitable hard materials are polymers such as polyesters, polypropylenes, polyethylene, polystyrene, ABS and polycarbonate; glass fibers and Plexiglas®.

In a preferred embodiment the cavity receiving the cannula extending from the connector is sealed with a membrane, preferably a self-closing membrane which is penetrated by the cannula when the infusion part is connected with the connector. Thus it is assured that drugs remaining in the infusion part do not flow out of the infusion part during movements in the disconnected state. As an alternative to a membrane a valve which manually can be opened/closed during connection/disconnection can be used.

In a preferred embodiment the infusion part and the connector have guiding means for guiding movement of the connector relative to the infusion part during connection or disconnection. Thus it is assured that the cannula does not penetrate into undesirable areas and that the cannula is positioned properly inside the cavity.

In one embodiment the infusion part comprises a first set of guiding means and the connector comprises a second set of guiding means adapted to fit with the first set of guiding means.

In one embodiment the guiding means are in form of at least one pin placed on the connector or the infusion part and at least one hole for receiving said pin placed on the other part.

In another embodiment the first set of guiding means are in form of at least one groove, preferably at least two grooves and the second set of guiding means are in form of at least one guiding fin, preferably at least two guiding fins.

In another preferred embodiment the infusion part comprises at first set of locking means and the connector comprises a second set of locking means said locking means being for locking/releasing the connector to/from the infusion part thus securing that the two parts do not unintentionally separate.

In a preferred embodiment the locking means are in form of retention devices.

In a preferred embodiment the retention devices are in form of at least two steps placed on either the infusion part or the connector and a matching carving in the other part. Preferably the step has a side with a triangular shape thus forming the step as a sloping hill. Preferably the retention devices are placed on the infusion part and the matching carvings are placed in the connector's arms.

In a preferred embodiment the first set of locking means are in form of a step and the second set of locking means are in form of two arms having a groove adapted to fit with said step.

In a preferred embodiment the connector is symmetrical both around the main plane of the connector and around the plane being perpendicular to the main plane and being parallel to the central axis, thus allowing the connector to be connected to the infusion part no matter which of the main sides is facing upwards. This results in a much easier operation of the infusion set.

In another preferred embodiment the arms of the connector have gripping means for getting a better grip of the connector. Examples of such gripping means could be but are not limited to rims, grooves, recesses, a roughened surface optionally of another material than the connector itself, preferably recesses are used. This results in a safer and more comforting operation of the infusion set since the risk that the fingers slip during handling resulting in unintended movements of the infusion part and the cannula is reduced.

In one embodiment of the invention the connector has at least one groove, preferably at least two grooves, placed in the guiding means of the connector, thus allowing the arms of the connector to rock relative to the guiding means. Hereby it is achieved that connection/disconnection can be performed in a manner which at the same time reduces the stresses in the material during the operation, eases the operation of the locking mechanism and reduces the patient's unpleasantness during the connection/release of the connector.

In another embodiment the infusion part has at least two cuttings creating at least two flaps. The created flaps can in an elastic manner move out of the main plane of the infusion part. Hereby the same advantages during connection/release as described above are obtained.

In a preferred embodiment the cannula of the connector is a hard cannula, preferably a metal cannula such as a steel cannula.

In another preferred embodiment the cannula of the connector is made of a plastics material and/or is being blunt.

In a preferred embodiment the infusion part and the connector are made from two different plastics materials, such as two different types of polypropylene.

In a preferred embodiment there is a visual difference in the toning of the connector and the base part of the infusion part. Hereby it is achieved that it is easier for the patient to see the separation line between the two units resulting in an easier operation of the locking mechanism.

In a preferred embodiment the cannula extending from the connector is extending from a central part of the connector and is placed in a withdrawn position relative to the front of the central part.

### BRIEF DESCRIPTION OF THE DRAWINGS

In the following the invention will be described in further details with reference to the figures.

FIG. 1 shows a first embodiment of the infusion set.

FIG. 2 shows a second embodiment of the infusion set.

FIGS. 3 a-f show different constructions of the micro needles.

FIG. 4 shows an embodiment of locking and guiding means.

FIG. 5 shows another embodiment of an infusion set.

### DETAILED DESCRIPTION OF THE DRAWINGS AND THE PRESENTLY PREFERRED EMBODIMENTS

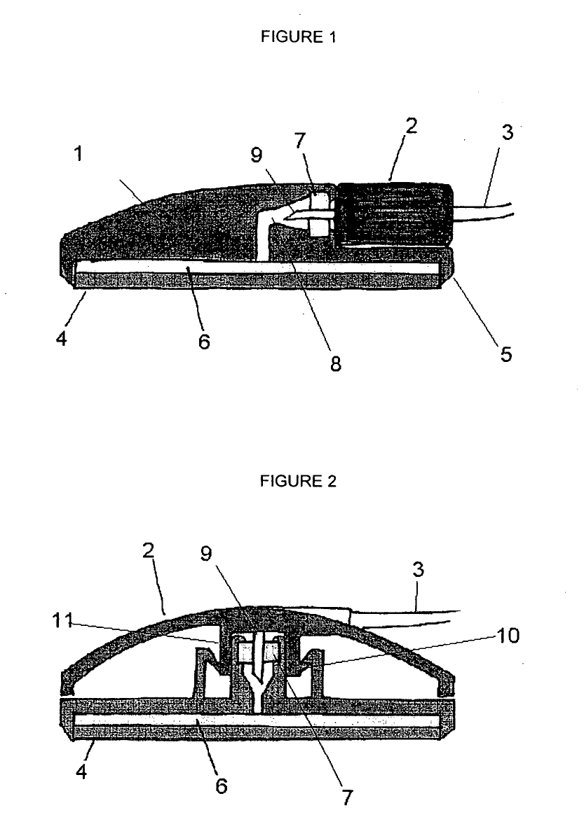

FIG. 1 shows one embodiment of the infusion set having an infusion part (1) and a connector (2) for connecting the infusions part to a not shown medical device through a conduit (3). The connector is fastened horizontally to the infusion part and has a cannula (9) which penetrates a membrane (7) and extends horizontally into a cavity (8) adapted to receive the cannula (9). A single conduct conducts the fluid from the cavity (8) to a chamber (6) which chamber is forming a coherent room above an array of micro needles. The chamber (6) can contain a material forming tracks in the coherent room thereby regulating the fluid pressure at the inlet of the micro needles. The chamber (6) is in fluid communication with the inlets of the multiple micro needles which in this embodiment are placed on a patch (4). Said patch is in this embodiment attached to the infusion part by means of two taps (5).

FIG. 2 shows a second embodiment of the infusion set comprising a connector (2) which is fastened vertically to the infusion part (1) and has a cannula (9) which penetrating the membrane (7) and extending vertically into the cavity (8). In this embodiment a possible elaboration of the guiding means (11) and the locking means (10) are shown.

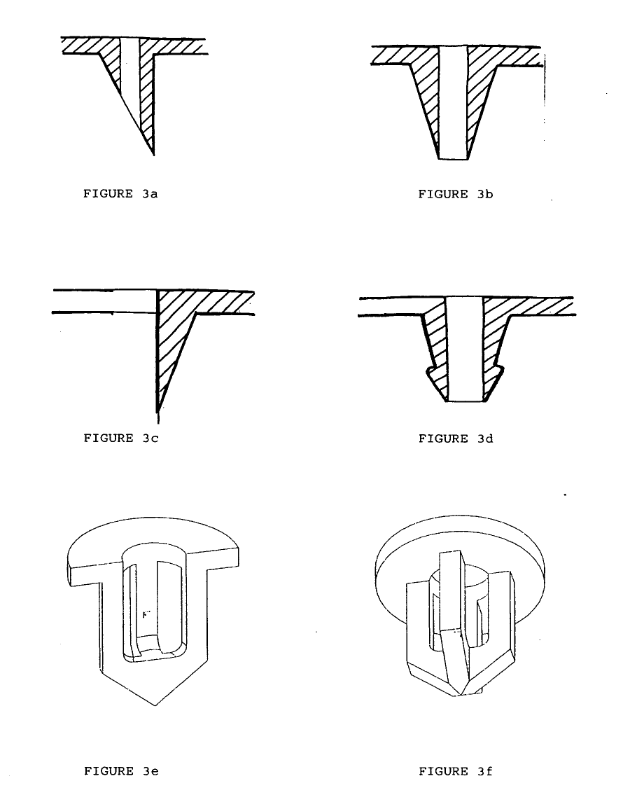

FIG. 3a shows a needle which has its outlet on the side. FIG. 3b shows a needle which has its outlet at the tip. FIG. 3c shows a needle which has its outlet next to the foot of the needle. FIG. 3d shows a needle with prickles. FIGS. 3e and 3f show two three dimensional views of a slashed needle.

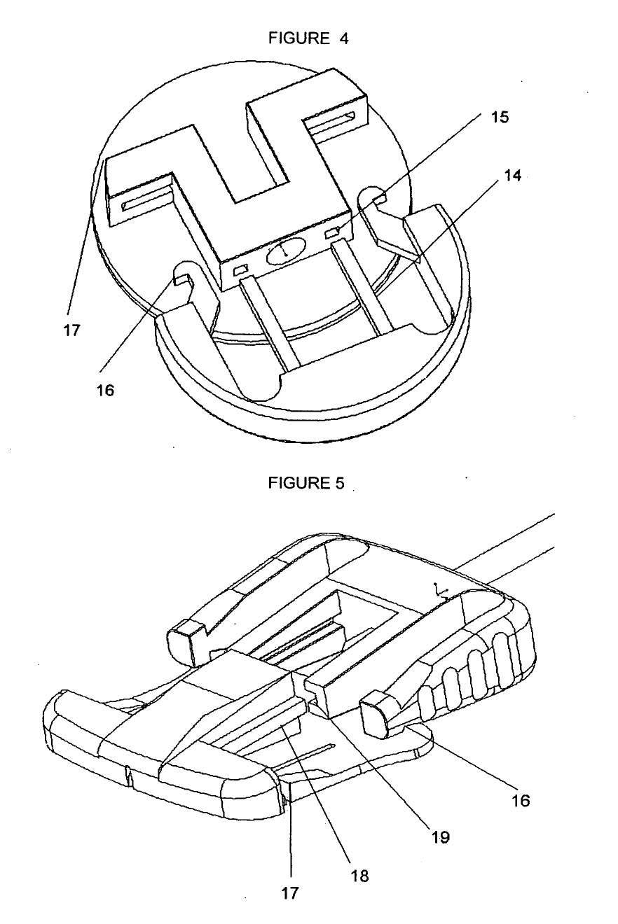

In FIG. 4 an embodiment of the guiding means is illustrated. In this embodiment two guiding pins (14) are placed on the connector and two holes (15) adapted to receive the guiding pins is placed in the infusion part. Further FIG. 4 illustrates one possible locking mechanism in which the connector has two arms each having a recess (16) and the infusion part has two steps (17) adapted to fit with the recesses.

In FIG. 5 is a second embodiment of the guiding means illustrated. In this embodiment the guiding means are in form of two guiding fins (18) and two grooves (19) adapted to receive said fins. Further is second locking mechanism shown said locking mechanism comprising four recesses (16) and two steps (17).

1. An infusion set comprising an infusion part (1) and a connector (2) being in fluid communication with a medical device, the infusion part (1) comprises multiple micro needles being in fluid communication with a chamber (6), and the chamber (6) is supplied with fluid from the medical device characterized in that fluid is conducted through a conduit which opens into the chamber (6), and the chamber (6) is in fluid contact with the inlets of all microneedles.

2. An infusion set according to claim 1, characterized in that the chamber (6) has rigid walls.

3. An infusion set according to claim 1, characterized in that the connector (2) is releasably fastened to the infusion part (1) and secures the connection between the infusion part (1) and the medical device as the conduit (3) is integrated with the connector.

4. An infusion set according to claim 1, characterized in that the connector (2) comprises a cannula (9) and that the infusion part further comprises a cavity (8) being in fluid communication with the chamber (6) and being adapted to receive the cannula (9) of the releasably fastened connector (2).

5. An infusion set according to claim 1, characterized in that the chamber (6) is filled with a transport material.

6. An infusion set according to claim 5, characterized that the transport material is selected from the group consisting of a hard material having grooves providing fluid communication through the chamber, a hard material having cross-millings, felt, a porous material, cotton, silicon or silicon oxides materials, blown materials and micro cellular urethane materials.

7. An infusion set according to claim 6, characterized in that the hard material is a polymer, glasfibres or plexiglass.

8. An infusion set according to claim 7, characterized in that the polymer is selected from the group consisting of polyester, polypropylene, polyethylene, polystyrene, ABS and polycarbonate; glass fibres and plexiglass.

9. An infusion set according to claim 1, characterized in that the chamber (6) has supporting members.

10. An infusion set according to claim 4, characterized in that the cavity (8) splits in multiple canals each being in fluid communication with the chamber (6) and having their length and diameter adjusted to each other in such manner that the fluid pressure at the inlet to the chamber essentially is the same.

11. An infusion set according to claim 1, characterized in that the micro needles are placed at a separate patch.

12. An infusion set according to claim 11, characterized in that the patch is attached to the infusion set by means of rivets, screws, by welding, by adhesives, by heat deformation or by having a patch slightly larger than the hole receiving it.

13. An infusion set according to claim 1, characterized in that the micro needles have one of the following shapes: a cone having a side outlet, a hollow needle with outlets on the side of the cylindrical part, a cone with outlet at the tip, a needle having an outlet at its attachment foot or next to its attachment point, a slashed needle, a needle having prickles and with outlet either at the bottom, at the side or at the point.

14. An infusion set according to claim 1, characterized in that the micro needles are made of a material selected from the group consisting of: pharmaceutical grade stainless steel, silver, platinum, titanium, nickel, iron, gold, tin, copper, chromium, alloys of previous metals, silicon, silicon dioxide, ceramics, acrylic materials, polymers and surface treated materials.

15. An infusion set according to claim 1, characterized in that the micro needles are made of a polymer selected from the group consisting of: polycarbonate, polymethacrylic acid, ethylenevinyl actetate, polytetrafluorethane and polyesters.

16. An infusion set according to any one of claims 1, characterized in that the micro needles are made of a biodegradable material.

17. An infusion set according to claim 16, characterized in that the biodegradable material is selected from the group consisting of: polymers of hydroxyl acids, polylactide, polyglycolide, polylactide-co-glycolide, polyanhydrides, pole(ortho)esters, polyurethanes, poly(butyric acid), poly(valeric acid) and poly (lactide-co-caprolactone).

18. An infusion set according to claim 1, characterized in that the infusion part further comprises an adhesive support.

19. An infusion set according to claim 18, characterized in that the adhesive support is a plaster.

20. An infusion set according to claim 1, characterized in that the area carrying the micro needles is raised from the lower surface.

21. An infusion set according to claim 1, characterized in that the area carrying the micro needles is the lower surface part having the largest distance to the main plane of the infusion part.

22. An infusion set according to claim 1, characterized in that the area carrying the micro needles is placed on the top of a hill.

23. An infusion set according to claim 4, characterized in that the cavity is covered by a self-closing membrane.

24. An infusion set according to claim 1, characterized in that the infusion part comprises guiding means.

25. An infusion set according to claim 1, characterized in that the infusion set comprises locking means for locking/releasing the connector to/from the infusion part.

26. An infusion set according to claim 1, characterized in that the connector is symmetrical both around the main plane of the connector and around the plane being perpendicular to the main plane and being parallel to the central axis.

27. An infusion set according to claim 1, characterized in that the connector has gripping means for getting a better grip of the connector.

28. An infusion set according to claim 1, characterized in that the infusion part further comprises a packaging.

29. An infusion set according to claim 28, characterized in that the packaging is a flange or a ring.

30. An infusion set according to claim 29, characterized in that the flange or the ring surrounds the needles.

31. An infusion set according to claim 1, characterized in that the infusion part further comprises a leak-detector.

32. An infusion set according to claim 1, characterized in that the infusion part further comprises an electrode.
